(19) United States
(12) Patent Application Publication
(10) Pub. No.: US 2019/0331701 A1
(43) Pub. Date: Oct. 31, 2019

(54) System and Method for Environmental Monitoring

(71) Applicant: Lonza Limited, Visp (CH)

(72) Inventors: Rex POLLEY, Exeter, NH (US); Anna GUEST, Visp (CH); Andrea PREUSS, Visp (CH); Hans HUMMEL, Visp (CH); Michael GOETTER, Mount Laurel, NJ (US)

(21) Appl. No.: 16/398,860

(22) Filed: Apr. 30, 2019

Related U.S. Application Data
(60) Provisional application No. 62/664,628, filed on Apr. 30, 2018.

(51) Int. Cl. G01N 35/00; G16H 40/20

(52) U.S. Cl. CPC G01N 35/0092; G16H 40/20; G01N 35/0099

(57) ABSTRACT

A system and method for monitoring an environment and performing analysis of collected samples are provided. The samples can be collected by personnel, a robot or a cobot, and the system may receive results of the monitoring/analysis and output information (e.g., alerts, reports, trends, forecasts) to environmental services, infection prevention personnel, and/or an electronic health/medical record system. If a contaminant is detected in the environment, appropriate personnel may automatically be alerted so that the contaminant can be eliminated/contained. The system may dynamically schedule the monitoring based on needs and ongoing changes in the environment, provide routing and work instructions to personnel, robots and/or cobots, and manage, maintain, and organize all information associated with the environmental monitoring so that information can be accessed, analyzed, and, if necessary, responded to, in real-time and/or in a timely manner, or where corrective actions need to be initiated as part of overall improvement plans.

### CROSS-REFERENCE TO RELATED APPLICATIONS

This application claims the benefit of U.S. Provisional Application No. 62/664,628, filed Apr. 30, 2018, the disclosures of which are expressly incorporated by reference herein.

### BACKGROUND AND SUMMARY OF THE INVENTION

Current practices within environmental monitoring revolve around a series of separate processes that can be manual and/or captured by a range of devices. But one or more of these various processes are typically implemented as separate and discrete systems in the environment, which can create inconsistencies in the reporting or alert of contamination due to, for example, processing delays with respect to each practice, overall inaccuracies and inefficiencies, or monitoring activities can be missed by ineffective scheduling, and data may be dispersed and disorganized, which may make it difficult to track and analyze the monitoring of the environment.

The present invention brings together some or all of these processes into a single system. The present invention also offers additional improvements by offering a menu of enhanced options where the user may use different analytical methods, techniques, and outputs, such as rapid diagnostics to improve accuracy and speed of testing, automation of all or some of the steps or processes or the like. The present invention also has the capability of setting up an alert system once these analyses are completed or if there is a problem conducting any step of the analysis.

The present invention relates to the monitoring of an environment, for example, a pharmaceutical or biological manufacturing environment, a hospital or other healthcare environment, testing environments, as well as environments that require regular monitoring for contamination, and monitoring of equipment and human processes within an environment. The equipment may include, for example, instruments used for operations, hospital beds, etc. Personnel and/or one or more self-navigating devices, such as robots or Human-Collaborative robots (hereinafter “cobots”), may be deployed in the environment to conduct monitoring activities, collect samples, and/or perform analysis on the collected samples, where the results of the analysis may automatically trigger an alert—for example of a contamination (or possible contamination)—in the environment.

For example, in a healthcare environment, such as a hospital, dirty surfaces and unclean equipment (medical or otherwise) may rapidly cause microbial contamination, which can lead to the infection of patients and/or healthcare personnel in the environment. Various practices for testing surfaces, equipment, and/or people, as well as monitoring hygiene critical processes, and alerting appropriate personnel of contamination may be implemented in the healthcare environment.

Thus, there is a need for a single, centralized system that is implemented in the healthcare environment accessible by its various users in real-time. There is also a need to integrate all of the various analytical techniques and monitoring activities in one system and to dynamically schedule the monitoring of the healthcare environment, collect samples, conduct assessments, perform analysis on those samples, and/or detect the presence of contaminants (e.g., microorganisms/pathogens such as bacteria, yeast, fungi, viruses) in the air, on surfaces, equipment, water, personnel, etc. and output information (e.g., dashboard, alerts, reports (including plotting of ‘hotspots’ on a map or site representation to define risk areas), trends, forecasts) to personnel and/or electronic health/medical record systems in a timely manner.

The integrated system of the present invention centralizes the information collected through various means into one central system for analysis and reporting. The information can be collected from a plurality of locations (e.g., a multi-site hospital or hospital with numerous off-site clinics) and provided to an electronic health/medical record system, which can be linked via bar code information. By collecting, analyzing and reporting the information as described above, successes and failures of equipment sterilization for specific patients can be provided and used to trace back for implementing corrective measures.

In one embodiment, robots may be used to automate certain processes. For example, robotic automation systems may be implemented in warehouses or factories or in any manufacturing setting to assist in the automation of assembly, sorting, moving items and inventory, etc. Moreover, “self-driving” vehicular robots may move bins or totes containing items from one location in a warehouse or factor to another location, as an alternative to using a conveyor system.

While the implementation of basic robotic functions to automate tasks in the manufacturing setting is known, automating certain tasks using robots, cobots and/or sensors, such as collecting samples and performing analysis of those samples for possible contamination in healthcare or other environments, potentially at the point of sampling, poses unique challenges, which are addressed by the present invention.

The system also encompasses cleaning after sampling. This could be done by personnel, robots or cobots. For example, testing that uses Replicate Organism Detection And Counting (RODAC) plates are growth promoting so the testing area would need to be cleaned after the sampling or testing is completed. Likewise, this would apply to any testing or sampling method that would result in contamination of any sort to the surface being examined.

### SUMMARY OF THE INVENTION

According to one or more aspects of the disclosure, a system for monitoring an environment, such as a healthcare environment, is provided in a single, centralized, and streamlined manner. Whether samples or monitoring data come from personnel, a robot or cobot, the system may receive the results of the analysis (along with other monitoring-related data) and output information (e.g., alerts, reports, trends, forecasts) to environmental services, facilities management, cleaning companies, infection prevention personnel, electronic health record (EHR)/electronic medical record (EMR) systems and/or governmental agencies. For example, if a contaminant is detected in the environment, appropriate personnel may automatically be alerted so that the contaminant can be eliminated or contained. Further, a backwards assessment of the audit record can be used to perform an investigation based on the analysis results if a patient acquires a healthcare-associated infection (HAI) while in a healthcare environment. For example, a sterilization record for a piece of equipment used by the patient can be evaluated during the investigation. This is made possible by the environmental monitoring system (e.g., MODA™ system commercially available from Lonza, Inc.) and its ability to trace equipment, instruments and recorded activities via a bar code scanning capability. The system may dynamically schedule the monitoring based on various needs and ongoing changes in the environment, and also provide routing and work instructions to personnel and/or robots and/or cobots, and further manage, maintain, and organize all information associated with the environmental, equipment and human monitoring so that information can be accessed, analyzed, and, if necessary, responded to, in real-time and/or in a timely manner. In one embodiment, all of these tasks are performed by one or more self-driving, self-navigating robots (and/or cobots) may be used to collect samples and/or perform analysis on the collected samples.

### BRIEF DESCRIPTION OF THE DRAWINGS

FIG. 1 illustrates an example system in accordance with one or more aspects of the present disclosure.

FIG. 2 illustrates an example robot in accordance with one or more aspects of the present disclosure.

FIG. 3 illustrates another example robot in accordance with one or more aspects of the present disclosure.

FIG. 4 illustrates an example chart including the various features related to an environmental monitoring system in accordance with one or more aspects of the present disclosure.

FIG. 5 illustrates an example robot performing sample collection and/or sample analysis in accordance with one or more aspects of the present disclosure.

FIG. 6 illustrates humans, sensors, robots and cobots in an example environment in accordance with one or more aspects of the present disclosure.

FIGS. 7A and 7B illustrate example flow charts in accordance with one or more aspects of the present disclosure.

FIG. 8 illustrates an example contact plate analysis in accordance with one or more aspects of the present disclosure.

FIG. 9 illustrates an example ATP measurement analysis in accordance with one or more aspects of the present disclosure

FIG. 10 illustrates a full environmental monitoring system in accordance with one or more aspects of the present disclosure.

FIG. 11 illustrates an example system in accordance with one or more aspects of the present disclosure.

### DETAILED DESCRIPTION OF EMBODIMENTS OF THE INVENTION

The present invention relates to the monitoring of an environment, including equipment and healthcare workers/cleaning personnel, that requires monitoring. This includes hospitals and healthcare environments, specialty manufacturing facilities, such as pharmaceutical and biological, medical device manufacturing facilities, and the like. As used herein a healthcare environment is any environment configured for treating or seeing a patient such as but not limited to a hospital, doctor's office, General Practitioner's surgery, dental office or clinic, outpatient treatment facility, nursing or palliative care facility, health clinic, dialysis clinic, military field hospital or tent, veterinary clinic or office, or other patient treatment setting.

In some aspects, self-driving and self-navigating robots and/or cobots may be deployed in the environment and configured to collect samples and/or perform analysis on the collected samples. In some aspects, personnel such as healthcare staff, sanitation staff, environmental services or facility management personnel, cleaning staff, or contractors, may be utilized in the environment and be instructed to monitor data, collect samples and/or perform analysis on the collected samples as well as manipulate and interact with a centralized system or other automated control system. In other aspects, personnel such as healthcare staff, sanitation staff, environmental services or facility management personnel, cleaning staff, or contractors, may work with a cobot in the environment and be instructed to collect samples and/or perform analysis on the collected samples as well as manipulate and interact with a centralized system or other automated control system. Regardless of whether collected via a robot/cobot or personnel, the results of the analyzed samples may be sent to or manually entered into a single, centralized system so as to automatically alert appropriate environment personnel of contamination within the environment in a timely manner and/or provide the information to other systems such as an electronic health record (EHR) system. To at least that end, the monitoring and/or cleaning of the environment may be automated (or semi-automated) and streamlined.

For example, certain areas of a healthcare environment, such as a hospital, may be monitored for contamination (e.g., microorganisms/pathogens such as bacteria, yeast, fungi, viruses). These areas of the hospital, which require varying degrees of sanitization, include operating rooms (OR), intensive care units (ICUs), patient rooms, nursing stations, and waiting areas. A waiting room, for example, does not require the same high degree of sanitization as an OR, ICU or even patient rooms. A system, which is implemented as a single centralized system, may be configured to: (i) dynamically schedule and plan the monitoring of data and collection of samples within the environment (e.g., surfaces, air, water, people, equipment), (ii) collect the samples and/or test and/or monitoring results, (iii) process and/or perform analysis the collected samples or assessment data, and (iv) automatically output or generate information based on the results of the monitoring (e.g., reports, alerts, trends, forecasts, dashboards, or other types of information), such as providing the information to EHR, EMR or other medical record systems, so that appropriate action can be taken in the event of contamination.

In one embodiment, computer-controlled, self-driving, and/or self-navigating devices (or any suitable autonomously guided vehicle, AGV) equipped with sensors, tools, collection and testing equipment, etc. may move about in the environment and collect various samples. These devices may be configured as robots (fully autonomous), cobots (semi-autonomous) or stationary sensors. A “co-bot” may be a device that is configured to assist a user (and is not necessarily fully autonomous) and may, for example, be physically moved by the user from one location to another location (as opposed to being self-driving). A sensor may be plugged into a single location and take continuous samples.

The collected samples may be processed and analysis on the samples may be performed. As will be described below, the analysis may be performed by the robots themselves or by personnel at the sampling location, or may be performed somewhere else, such as by an analytics device or technician in a lab in the environment. The robot, cobot or sensor may communicate with the system via a network to at least: (i) receive routing and/or work instructions and (ii) transmit the results of the analysis on the collected samples. In some aspects, the data can be collected offline and downloaded en-masse once in-range of the centralized system or other collection point such as a wireless gateway in communication with the centralized system or network. The system may share the results with other computing devices connected to the network or through manual download or the like. In embodiments, a plurality of robots and/or cobots and/or sensors may be deployed in the environment, each of which may be configured to collect different samples, data, and/or perform analysis on the samples using different analytical techniques.

Various systems, methods, and/or techniques may be used to perform analysis on the collected samples. In one example, a visual system (e.g., observation and/or use of fading dyes) may be implemented to “watch” how effectively healthcare personnel are cleaning the surfaces and whether the cleaning practices adhere to cleaning protocol, procedures, and meet various requirements, e.g., CDC requirements, hospital standard operating procedures, and/or other requirements. In another example, an ATP measurement system may be used to measure and detect the presence of adenosine triphosphate (ATP), which is a compound that is present in all living tissue, on various surfaces and equipment. In yet another example, a system may be used where surfaces and medical equipment may be “pre-marked” with marking agents, such as fluorescence gel, and analyzed whether the marking agent on the surfaces and medical equipment has been entirely cleaned off. In a further example, a lab-based system may be used where the surfaces and medical equipment are swabbed and tested for the presence of contaminants, microbial counts, or pathogens, which again may reveal whether cleanliness requirements are being met in the environment. An example of surface testing is contact plate analysis. In another example, an air detection system may be used to detect how clean (or dirty) the air may be. In another example, a water analysis and detection system may be used to assess particular attributes of a water source (e.g., presence of specific microorganisms/pathogens). For example, testing of facility potable water—temperature, Legionella, Pseudomonas aeruginosa, as well as the various use patterns that are tested from an Infection Prevention & Control (IPC) perspective such as endoscope rinse water, renal dialysis water, hydrotherapy pools, etc. can be performed and the results collected by the system. IPC audits are facilitated by the monitoring and sampling by including, for example, checklists to monitor other processes or indicators such as (i) Intravenous (IV) catheter insertion, (ii) wound dressing change, (iii) multidrug resistant organism (MDRO) transmission-based precautions, (v) process of disinfecting and sterilizing equipment/instruments, and (vi) a record of cleaning within the ward environment. Further, collected temperature, humidity, air flow monitoring and/or other physical chemical parameter data can be compared against predetermined limits.

In yet another example hand hygiene monitoring is provided. In aspects of the present disclosure, the monitoring of hand hygiene includes a range of solutions from basic methods, such as self-reporting, direct human observation, swabbing of healthcare workers' hands, video observation up to fully integrated systems where personnel wear sensors that may be networked with dispensers and patient locations, etc. For example, observation of how a cleaner conducts their duties while cleaning or observation checklists to assess how well a healthcare worker addresses hand hygiene while attending to patients or performing other hygiene critical tasks such as changing a catheter may be used. Other options may include a device where personnel can place their hands under a UV light that can then detect how well they have washed their hands, and the system may be configured to give instructions on proper handwashing technique. To at least that end, the hand hygiene monitoring feature of the system is to improve overall compliance with hand hygiene program.

By way of example, the system integrated into the environment may include an automated or semi-automated device that samples pathogens on hands and/or skin of healthcare personnel or patients within the environment, samples of which would be collected and analyzed to provide real-time data evaluation and feedback on any contamination by the system. Accordingly, recommendations as to how to mitigate any contamination or outbreak of pathogens may be provided by the hand hygiene monitoring feature of the system.

In one embodiment, personnel could wear transmitters on their persons, such as part of their employment credentials or “ID badges.” Sensors may be placed at handwashing stations, hand sanitizing stations, or other decontamination stations. The sensors would collect data on which employee was present, the duration of that employee's time, whether soap was dispensed, the length of time the water was on, and other salient data to determine the appropriate level of hand hygiene. This data would then be sent back to the system of the present invention for analysis and reporting.

In another embodiment, environmental services or cleaning personnel or could manually input, or the hand sanitizer and hand soap dispensers could automatically transmit, product usage information to the system such as volume of product per dispenser. The sensors in the hand hygiene dispensers would collect data on the amount of product dispensed per dispenser over a given period of time, or log when the dispenser is refilled with product. This data would then be sent back to the system of the present invention for analysis and reporting of indirect hand hygiene compliance. Other examples include novel rapid detection methods (e.g., biomarker based), quick Polymerase Chain Reaction (qPCR), next generation sequencing (NGS), and whole genome sequencing (WGS). The above described systems, methods, and/or techniques may be fully automated (e.g., via robots) or semi-automated (e.g., via cobots) or through personnel, or some combination thereof.

In another embodiment, data and information associated with the collected samples and the performed analysis may be sent to and/or dynamically processed by the system for further analysis or output. For example, appropriate personnel in the environment may be alerted (e.g., text, e-mail, paging messages) of a contamination in a particular room or section of the environment. The alert may be sent to the personnel's mobile computing device (e.g., smartphone, laptop, tablet computer). In a further example, a dashboard interface may be generated, and a user, such as hospital personnel, may view reports, alerts, trends, and/or maps of “hot spots” related to the monitoring of the environment. In a further example, the data could be forward integrated into another IT system such as an electronic medical/health record. The system may also present, link, or associate various products or services to personnel in order to eradicate the reported contaminations or it can link cleaning/sterilization data from instruments and equipment to specific patients via integration into patient EHR systems. For instance, a particular product that can eradicate a specific pathogen may be presented to hospital personnel, if that specific pathogen was detected in the environment. Further, where a rotation of cleaners, disinfectants or sanitizers are used, the system can enforce, by manual or bar-code entry, that the proper product and concentration are being applied for that day, time, or pathogen.

The system implemented in the environment may “learn” over time how to optimally monitor the environment and dynamically schedule and prioritize certain tests and monitoring activities at particular locations of the environment based on various things, such as history and frequency of contamination and occurrences of non-compliance with standard operating protocols. The system may also dynamically generate optimal navigation routes for the robots and/or cobots. It may be understood that the system may also seamlessly integrate, receive, process, and/or output information from other types of testing equipment or devices within the environment (new or already existing), such as bench-top equipment, air sampling equipment or other suitable equipment in labs, stationary computers or sensors configured to perform visual observations. It may be understood that the system may also seamlessly integrate, receive, process, and/or output information from a laboratory information management (LIMS) system.

As will be further described below and will be apparent in the disclosure, one of the numerous advantages of the present invention is that there can be a single, centralized system implemented in the environment that regulates the monitoring, collection of samples and monitoring assessments, and analysis of samples from the environment, and dynamically and automatically generates an output, such as a real-time alert to appropriate environment personnel if a contamination is detected. The system may also be configured to autonomously perform analysis on those collected samples itself. For example, the system 100 may be integrated with various laboratory equipment, such as various types of sample processing, sample testing, and/or sample analytics equipment, so that it can analyze in real-time (and on-the-spot) the collected samples in order to determine whether the environment contains a contamination. The system can communicate with robots and/or cobots and/or sensors and/or various laboratory equipment, such as various types of sample processing, sample testing, and/or sample analytics equipment deployed in the environment, which are autonomously or semi-autonomously configured to carry out the above described sample analytics. In at least that regard, the speed at which results are obtained is significantly increased. Moreover, the centralized system allows real-time reporting of contamination (or possible contamination) so that it can be handled by appropriate personnel, or even a robot or cobot, in a timely fashion. The system may also produce reports, trends, forecasts, and other suitable types of analyses for environment personnel to further review and analyze in order to maintain a clean environment. The system 100 may have full control over how the warning is output. For instance, the system may cross-check the results sent by these devices with other system data to verify that the contamination does exist and issue a warning to appropriate personnel, while the system transmits similar alerts to other personnel in the hospital via mobile devices, computers, etc. The system may also present, link, or associate various products or services to personnel in order to eradicate the reported contaminations or it can link cleaning/sterilization data from instruments and equipment to specific patients via integration into patient EHR systems.

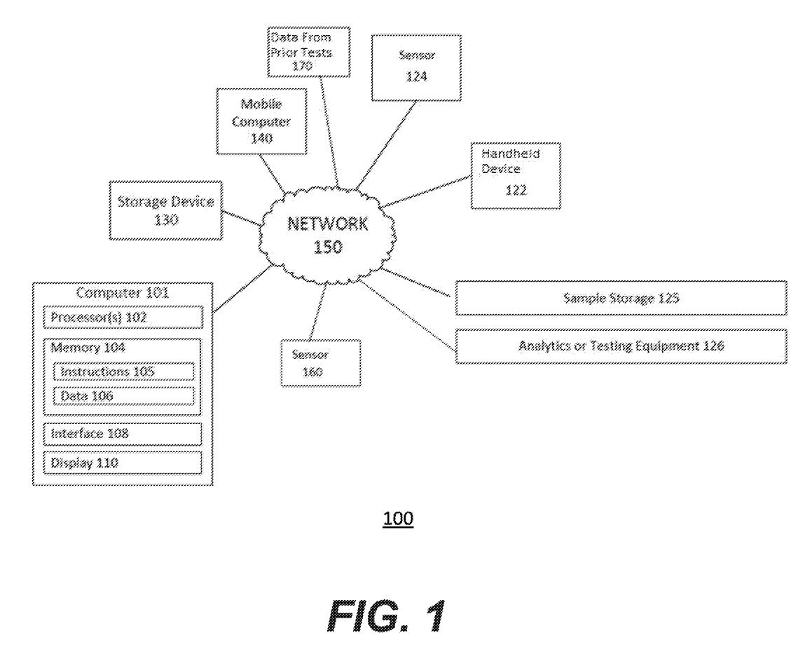

FIG. 1 illustrates an example system 100, which may be implemented within an environment, such as a hospital, a pharmaceutical or biomanufacturing facility, a water testing facility, or the like in accordance with one or more aspects of the invention. As shown, the system 100 includes at least a computer 101, a storage device 130, a mobile computer 140, sensors 124, 160, a handheld device 122, sample storage 125, analytics or testing equipment 126, which may all connect to a network 150 (as depicted by the dashed lines) and communicate with each other or other devices either on the same network or other networks. Moreover, data from prior tests 170 may be transmitted via the network 150.

For example, computer 101 includes one or more processors 102, memory 104, e.g., permanent or flash memory (which includes instructions 105 and data 106), an interface 108, and a display 110.

Processor 102 may instruct the various components of the computer 101 to perform tasks based on the processing of certain information, such as instructions 105 and/or data 106 stored in the memory 104. The processor 102 may be any standard processor, such as a central processing unit (CPU), or may be a dedicated processor, such as an application-specific integrated circuit (ASIC) or a field programmable gate array (FPGA) or an industrial process controller or the like.

Memory 104, whether permanent or flash, may be any type of hardware (e.g., ROM, RAM, CD-ROM, hard drive, write-capable, read-only, etc.) configured to store information accessible by the processor 102, such as instructions 105 and data 106, which can be executed, retrieved, manipulated, and/or stored by the processor 102. The instructions 105 stored in memory 104 may include any set of instructions (e.g., “steps” or “algorithm” associated with software) that can be executed directly or indirectly by the processor 102. The data 106 stored in memory 104 may be retrieved, stored or modified by the processor 102, for example, in accordance with the instructions 105.

Interface 108 may be a particular device for interfacing with the computer 101 (e.g., a field-mounted instrument, processor-to-processor communication, keyboard, mouse, touch sensitive screen, camera, microphone, etc.), a connection or port (e.g., data port, USB, zip drive, card reader, CD driver, DVD drive, etc.), and/or software (e.g., graphical user interface) that allows the reception of information and data.

Display 110 may be any suitable type of device capable of communicating data to a user, such as liquid-crystal display (LCD), light emitting diode (LED), and plasma screens.

Sensors 124, 160 may include image sensors, laser sensors, infrared sensors, touch-sensitive sensors, acoustic sensors, handheld ATP machines, plug-in air sampling devices, water sensors or any other type of sensor. For example, one or more of the sensors 124 may be configured as to collect information from the environment (such as air or water) and transmit that to the system 100. In some embodiments, the sensor 124 may have a badge scanner in order to scan personnel badges in the environment. In at least that regard, the sensor 124 may be able to determine at least two things: whether the personnel is or is not supposed to be at that particular location of the environment and also whether the personnel is contaminating the environment, and if so, alerting the personnel of such contamination (e.g., telling the personnel to wash his or her hands, telling the personnel that he or she needs a refresher on handwashing practices).

In further embodiments, sensors 124, 160 may be hand hygiene related sensors, e.g., sensors that can be worn on or by the personnel, sensors incorporated into hand washing or hand hygiene stations (such as hand sanitizer dispensing stations, dispensing equipment, smart dispensers, touch and/or no-touch dispensers, automated and/or manual dispensers, etc.), image sensors (such as cameras) for visually observing hand hygiene, and the like. It may be understood that any other types of sensors or devices that collect data can be used. From these sensors, system 100 may be able to collect data related to hand hygiene of environment personnel, patients, visitors, people, etc. and evaluate the collected data. As described above, the system may be able to generate reports, alerts, feedback, recommendations, evaluations, etc. to various personnel via, for example, a dashboard, in order to allow real-time monitoring and personnel engagement. In turn, for instance, this promotes hygiene compliance and improved database management and networking of the monitoring information of hand hygiene. By way of example, a sensor (e.g., sensor 124) provided at a hand washing station may send collected data to the system, where the system can detect and/or determine that a hospital employee has not washed his or her hands properly. The system may provide a dashboard alert on the employee's smartphone instructing to follow proper handwashing procedures.

The storage device 130 may be configured to store a large quantity of data and may also be configured to transfer such data when requested or accessed by other components of the system 100, either through the network 150 or otherwise. For example, the storage device 130 may be a collection of storage components, such as ROM, RAM, hard-drives, solid-state drives, removable drives, network storage, virtual memory, multi-leveled cache, registers, CD, DVD, etc. In addition, the storage device 130 may be configured so other components of system 100, such as the computer 101, and/or mobile computer 140 can have access and provide data to it.

The mobile computer 140 may be a laptop (or any type of computer that is portable or mobile, such as an Ultrabook, smartphone, PDA, tablet computer, a wearable computing device, etc.) and also include components similar to the computer 101. The mobile computer may also have one or more processors, memory, user interfaces, wired or wireless network connection hardware, and other types of components associated with a mobile computing device. In one or more embodiments, the mobile computer 140 may also be configured to execute software supported by computer 101 and communicate with other components of system 100 via network 150. Handheld device 122 may be configured similarly to the mobile computer 140. By way of example, if a contaminant is detected in the environment, the handheld device 122 may receive an alert and the user of the handheld device 122 may be informed exactly where the contaminant is located and/or how it can be eradicated.

Network 150 may be any suitable type of network, wired or wireless, configured to facilitate the transmission of data, instructions, etc. among the components of the system 100. For example, the network 150 may be a local area network (LAN) (e.g., Ethernet or other IEEE 802.03 LAN technologies), Wi-Fi (e.g., IEEE 802.11 standards), wide area network (WAN), virtual private network (VPN), global area network (GAN), or any combinations thereof In embodiments, the network 150 may be installed and implemented in the environment, such as a hospital, and may connect to other networks, such as the Internet, cloud-based networks, or networks in other environments, for example, medical record systems such as an EHR and EMR systems.

It may be understood that the above-described computer 101 may be a laptop, a desktop computer, server computer (which may be rack-mounted), “cloud” computers, virtual computers, or any device capable of processing data and/or instructions and transmitting and/or receiving data. Moreover, it will be understood by those of ordinary skill in the art that any of the computing devices or computers illustrated in FIG. 1 may actually include multiple processors, memories, instructions, data or displays that may or may not be stored within the same physical housing. And although some of the components of FIG. 1 are connected to the network 150, it may be understood that the components may also be connected to each other, in any suitable combination. It may also be understood that network 150 may include a plurality of sub-networks that may be connected to each other, and/or the network 150 may be connected to other external networks.

In embodiments, the system 100 may be implemented as a single, centralized system in the environment to facilitate the monitoring of the environment. The “brains” of the system may be carried out by one or more computers, such as computer 101, which may be server computers that reside physically at the environment, or elsewhere. The computer 101 may provide instructions via antennas installed throughout the environment.

Additionally, information and data may be received by the computer 101 from the sensors and personnel operated devices in the system, which may include data related to the various samples that have been collected and analyzed from the environment. In examples, the system may send alerts to various computing devices, such as the mobile computer 140 or handheld device 122 (which may belong to hospital personnel). Moreover, the system may account for Wi-Fi gaps or equipment interference with Wi-Fi communication, and the system may also be configured to operate offline. In at least that manner, the system is robust. As will be further described below, the “brains” of the system may regulate various tasks, such as generating, maintaining, and updating routing and navigation instructions, dynamically setting up schedules for various types of testing, keeping track of infected or contaminated locations and/or people in the environment, and dynamically updating its respective database and any and all information related to the monitoring of the environment. The system may also receive data from prior tests 170 and/or data from analytics or testing equipment 126 having performed tests on samples stored in sample storage 125 to perform all of the aforementioned features.

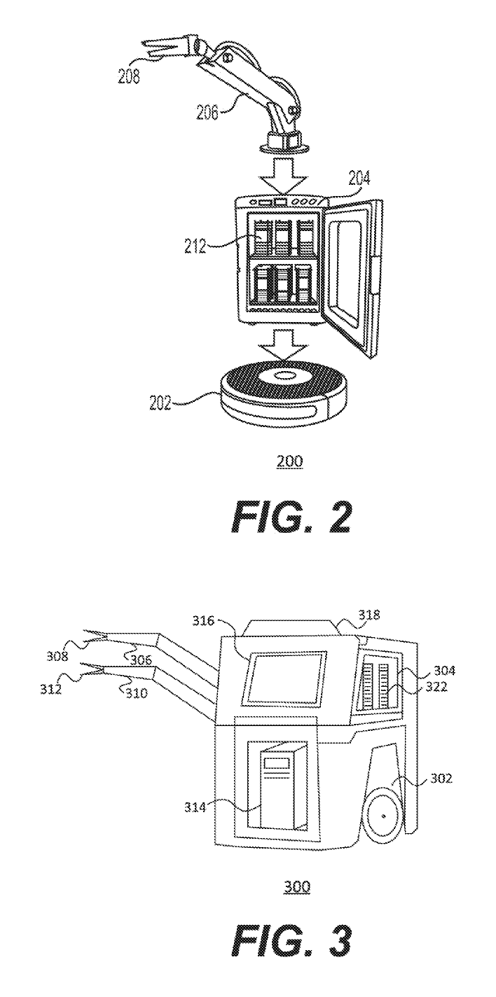

FIG. 2 illustrates another embodiment of the invention, which includes a robot 200 in accordance with one or more aspects of the present invention. As shown, robot 200 includes at least three separate components: (1) an autonomous, self-driving and self-navigating platform 202, (2) an incubator or other sample storage and/or processing equipment 204, and (3) at least one robotic arm 206 with an end effector 208. The equipment 204 may be mounted on top of the autonomous platform 202, and the robotic arm 206 may be mounted on top of equipment 204, as depicted in FIG. 2. It may be understood that robot 200 may include other various types of components, such as testing equipment, tools, docking station, scanner, radio frequency devices (e.g., for reading RF badges), equipment to locate the robot, which may also be RFID, batteries, etc., which are not shown in FIG. 2. Further, the robot 200 could actually place RFID markers to indicate a site for another robot or person to find and act upon. It may be further understood that that the components of the robot 200 may be arranged or assembled in numerous different ways, and not limited to the configuration or number of components as shown in FIG. 2.

In one example, the autonomous platform 202 may move about within an environment, such as a hospital, from one location to another location to the next location, etc., and collect various samples within that environment. For instance, the robot 200 may collect samples from a surface in a patient room, move to an operating room and collect samples from surfaces in the operating room, and move to a reception area of the hospital and collect surface samples from the reception desk.

Robot 200 may additionally have the ability to sterilize its treads, wheels or other aspects that come in contact with the environment. For example, robots may have built in self-sanitizing devices, products, or cleansers. Additionally, robots and cobots may have the ability to transfer samples collected to other robots, cobots, computers, or personnel for further processing, thereby reducing the risk of possible cross-contamination. For example, a robot tasked with taking samples from the infectious disease isolation room would transfer samples to another robot, cobot, or personnel without leaving the isolation room and causing any potential contamination.

Robot 200 may include one or more computing devices, various sensors, and at least one communication interface, which allows the robot 200 to communicate with the centralized system via a network and corresponding antennas that may be installed in the hospital. The robot 200 may receive routing instructions via the communication interface and the one or more computing devices may control the autonomous platform 202 in the hospital according to the received routing instructions. Similarly, robot 200 may also receive work assignments, which may include instructions regarding which surfaces to sample, which locations in the hospital, the number of samples to collect, how to process those samples, how to perform analysis on the samples, etc.

Robot 200 may be configured to collect microbiological samples from various surfaces by using plates 212, such as Replicate Organism Detection And Counting (RODAC) plates. RODAC plates may be used and available for different growth media, such as for determining the amount of, for example, Staphylococcus aureus or determining the amount of Enterobacteriaceae. The robot 200, using its robotic arm 206 and its end effector 208, may swab the surface and place the sample on plates 212, or contact the plates 212 to the surface to collect the sample. The robotic arm 206 may place and store the plates 212 in the equipment 204 until they can be processed and analyzed. For instance, the autonomous platform 202 may drive the robot 200 to an assigned location (e.g., lab, autoloader, station, etc.), where it can, for example, remove the plates 212 and place them into an analytics machine for analyzing the collected samples. Once the plates 212 have been removed, the robot or cobot would clean and disinfect the area or alert personnel to perform this task.

In some examples, the incubator or other sample storage and/or processing equipment 204 may be modular, so if an incubator, such as sample storage and/or processing equipment 204, is transferred to a lab, a new incubator may be attached by or to the robot for the next assignment or task. Robots and cobots could also have the ability to start numerous other analyses while the sample is being transported to speed up processing time.

FIG. 3 illustrates a robot 300 in accordance with one or more aspects of the present invention. Similar to robot 200 illustrated in FIG. 2, robot 300 incorporates various components, such as an autonomous, self-driving, self-navigating base 302 (not externally shown), an incubator 304, robotic arm 306 with end effector 308, robotic arm 310 with end effector 312, computing device 314, display interface 316, and communication interface 318. It may again be understood that robot 300 may include many other types of components and may be arranged or assembled in a different manner than the configuration shown in FIG. 3.

Robot 300 may not only be configured to collect different types of samples and assist appropriate personnel to perform the analysis of the collected samples, but robot 300 may also be configured to autonomously perform analysis on those collected samples itself. For example, robot 300 may be equipped with various laboratory equipment, such as various types of sample processing, sample testing, and/or sample analytics equipment, so that it can analyze in real-time (and on-the-spot) the collected samples in order to determine whether the environment contains a contamination. RODAC plates 322 stored in the incubator 304, for instance, or other collected samples, may be reviewed and analyzed by the robot 300 in real-time or near-real-time as soon as possible after sampling and/or arrival in a laboratory.

Upon analysis, the robot 300 may display the results of its analysis on the display interface 316 and may either indicate that the environment is free from contamination or warn appropriate hospital personnel that a contamination is present in the environment so that the contamination can be properly eradicated. In either situation (contamination or no contamination), the centralized system may store and maintain the results. Moreover, in some examples, the centralized system may have full control over how the warning is output from/to the robot 300. For instance, the system may cross-check the results sent by the robot with other system data to verify that the contamination does exist and instruct robot 300 to issue a warning to appropriate personnel, while the system transmits similar alerts to other personnel in the hospital via mobile devices, computers, etc.

In other examples, the robot 300 may be equipped with cleaning equipment to eradicate the contamination itself. Moreover, the results of the analysis may be transmitted and/or uploaded to the centralized system via the communication interface 308 for further review and analysis, if necessary, and/or for data collection and storage, e.g., time, place, type of contamination, etc.

Based on the sophistication of the analytics that the robot 300 performs, more robotic tools, equipment, and/or sensors may be added to the robot. As shown in FIG. 3, robot 300 has two different robotic arms 306 and 310 to simultaneously perform different tasks. For example, robotic arm 306 may be configured to handle the RODAC plates 322 for surface sampling, and robotic arm 310 may perform surface swabbing for ATP measurement, or perform other types of analytical tests, such as rapid detection methods, etc.

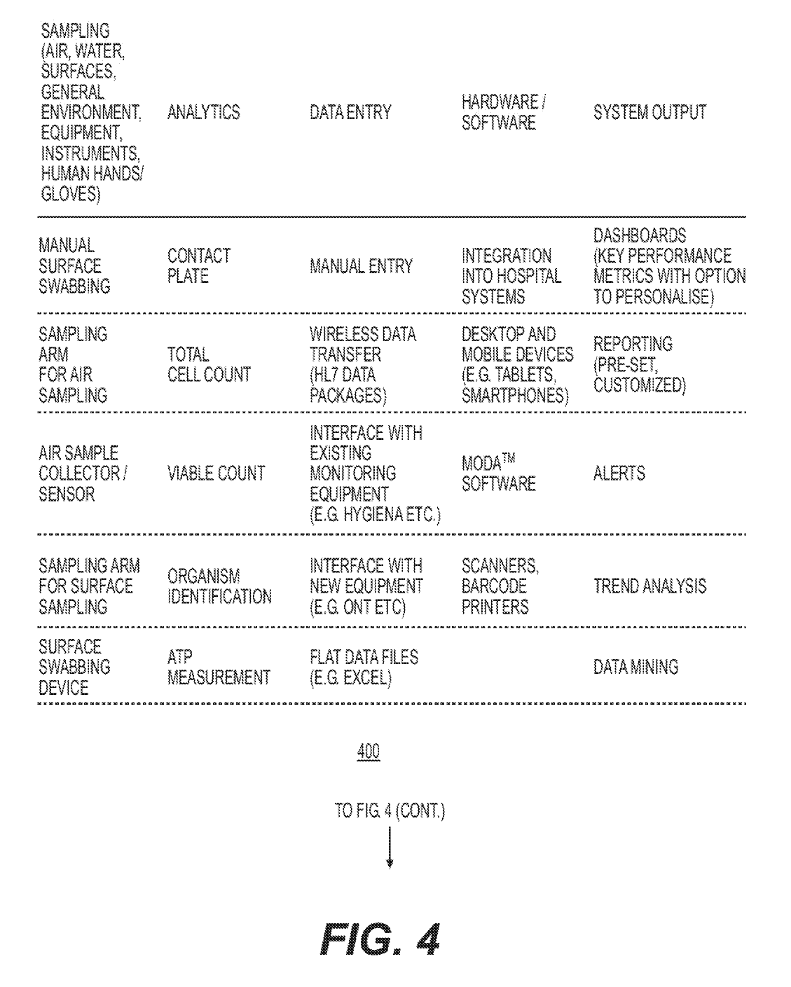

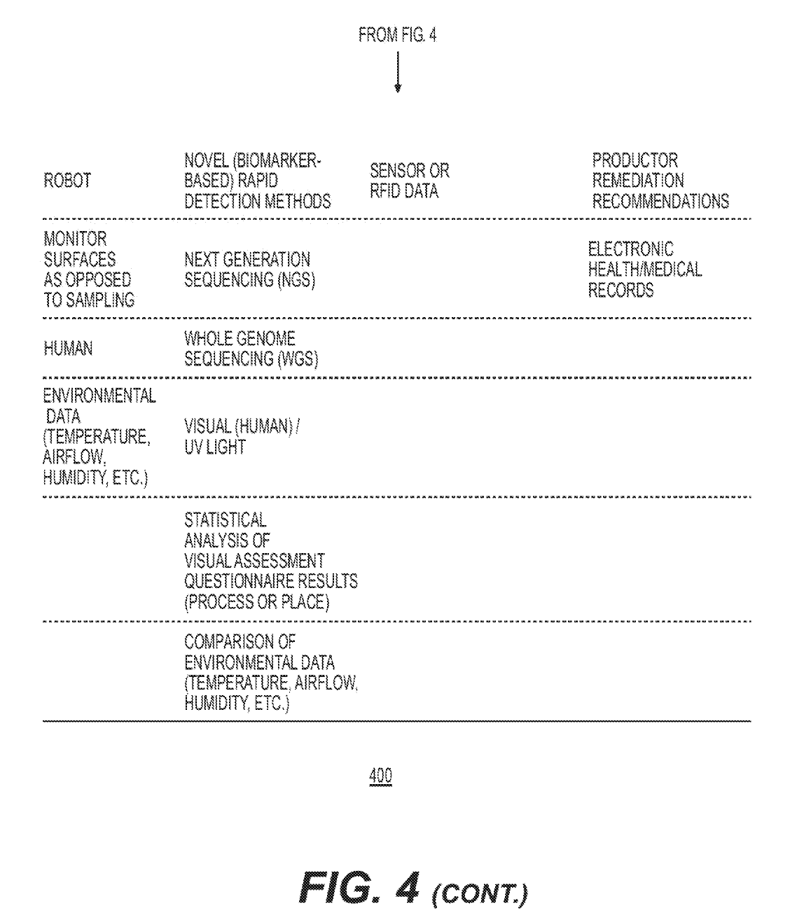

FIG. 4 illustrates a chart 400 containing various aspects with respect to environmental monitoring in accordance with one or more aspects of the present invention.

In embodiments and as discussed above, a robot, such as robot 200 or robot 300, may be configured with various sampling and/or monitoring equipment in order to sample and monitor numerous things (e.g., air, surfaces, water, humans, hospital equipment) within a particular environment, and may communicate with computer 100 via network 150 during the course of sampling and/or monitoring. For example, a surface may be swabbed by the robot using one or more robotic arms, a sampling arm may sample the air in the environment, an air sample collector or sensor configured in the robot may also sample the air, a dedicated sampling arm can sample a surface, image sensors in the robot may visually monitor the surfaces (as opposed to sampling), and the robot may monitor and/or sample humans within the environment.

The sampling and/or monitoring may be fully automated by the robot. In other words, environment personnel do not have to intervene in carrying out the sampling and/or monitoring process. Alternatively, using cobots (as opposed to robots), one or more of the different aforementioned sampling and/or monitoring techniques may be performed by the environment personnel. For example, the personnel may manually swab a surface and place the sample on or in one or more cobots, which can carry the sample to a lab for further analysis and processing. In at least that regard, only certain processes are automated (or semi-automated). Accordingly, cobots may be a self-driving cart and may not have the robotic arms, sensors, etc. integrated into robots.

As described above, analysis may be performed on the collected samples. Analytics include but are not limited to contact plate, total cell count, viable count, organism identification, ATP measurement, novel (e.g., biomarker-based) rapid detection methods, organism separation by electric field application, quick Polymerase Chain Reaction (qPCR), nanopore sequencing for direct, electronic analysis of single molecules, Next Generation Sequencing (NGS), Whole Genome Sequencing (WGS), visual/UV light/FTIR, comparison of collected temperature, humidity, air flow monitoring data and/or other physical chemical parameter data can be compared against predetermined limits, etc. In some aspects, for example, commercially available sequencing devices such as those available from Oxford Nanopore Technologies Limited can be utilized.

In examples, some or all portions of the analysis of a sample may be performed at a particular location (e.g., a lab, station, location of autoloader, etc.). Robot 200 in FIG. 2, for example, may collect the sample, but transfer the collected sample to that location for analysis and, potentially, for uploading either to the robot or to another data repository. Additionally or alternatively, some or all portions of the analysis of a sample may be performed by the robot itself using on-board testing or analytics equipment. Additionally, the robot could also ‘sub-sample’ in-place or while in transit to the lab, where a single sample could be parsed to be processed by several different analysis machines.

As shown in FIG. 4, data may be acquired and managed in various ways with respect to the system-side. FIG. 4 also shows how data may be acquired and managed in various ways the robot/cobot/sensor-side, where used according to some aspects of the present invention. Data may be manually entered (e.g., individual, bulk), or transmitted or received via wireless data transfer (e.g., HL7 data packages), data may be acquired, transmitted, managed, etc. via various interfaces (e.g., existing monitoring equipment, new monitoring equipment, bench-top equipment), and data, such as flat data files (e.g., excel), may be stored locally in computing devices connected to the centralized system and/or in large databases connected to the centralized system.

Both hardware and software aspects of the system may be integrated into hospital systems, they may include desktop computers, mobile computers, tablet computers (e.g., tablets, smartphones) that can be used with the system, and may include software for seamlessly operating and running the centralized system (e.g., MODA™ informatics software products commercially available from Lonza, Inc.). For example, software systems such as MODA™ software commercially available from Lonza, Inc. can be configured to guide human workflows to enforce that optimal or otherwise predetermined materials are used in the right place at the right time.

And as further illustrated in FIG. 4, system outputs include dashboards (e.g., key performance metrics with options to personalize), reports (e.g., preset, customized), alerts, trend analysis, data mining, output file for forward integration into other systems such as EHR, etc. These outputs, such as alerts, may be transmitted to various computing devices that are connected to the system, such as mobile devices, tablet computers, laptops, etc. that belong to hospital personnel, and/or may be sent to the robots and/or cobots to also issue alerts or allow personnel to access and view output data on the robot itself, for example, on display interface 316 of robot 300 in FIG. 3.

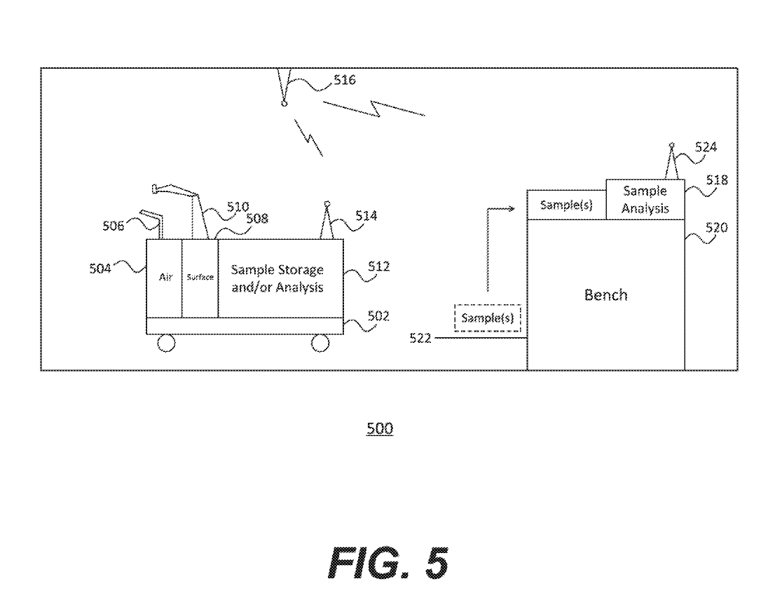

FIG. 5 illustrates an example of a robot 502 monitoring an environment 500 in accordance with one or more aspects of the present invention. Environment 500 may be a healthcare environment, such as a hospital, a manufacturing facility or a water testing facility. It may be understood that robot 502 may be also be a cobot in other examples.

As illustrated, robot 502 may include an air sampling unit 504 with air sampling arm 506, a surface sampling unit 508 with a surface sampling arm 510, sample storage and/or analysis unit 512, and a Wi-Fi antenna 514. Moreover, the environment 500 includes a Wi-Fi antenna 516, which transmits instructions to robot 502 via Wi-Fi antenna 514 regarding scheduling, routes, integration, workflow, analytics, visualization, etc. For example, routing instructions (in particular, how the robot 502 should navigate the environment) may be transmitted from Wi-Fi antenna 516 to 514, and robot 502 executes those routing instructions. And based on workflow instructions, in a further example, robot 502 may use the air sampling unit 512 to collect samples of the air (via the air sampling arm 506) and use the surface sampling unit 508 (via the surface sampling arm 510) to collect samples of various surfaces in the environment.

In one embodiment, the collected air, water and surface samples may be stored and analyzed by the sample storage and/or processing unit 512. Analysis may be performed using any one of the techniques or methods described above. During or after analysis, the robot 502 may transmit data associated with the analysis to a centralized system from Wi-Fi antenna 514 to Wi-Fi antenna 516. In at least that regard and as previously described, the robot 502, itself, may do both the collecting of the samples and the analysis thereof in real-time and on-the-spot.

In an alternative embodiment, the collected air, water and surface samples may be stored by the sample storage and/or processing unit 512. The robot 502 may transport the collected samples and deposit them to a sample analysis unit 518 sitting on top of a bench 520. The bench 520 may have one or more lifts 522 that may mechanically move the sample up to the sample analysis unit after the robot 502 places the sample(s) on the one or more lifts 522. Alternatively, robot 502 may directly transfer samples to sample analysis unit 518 and/or bench 520 as applicable. Similar to the storage and/or analysis unit 512, the sample analysis unit 518 may perform the analysis using any one of the techniques or methods described above. Moreover, the results of the analysis may be transmitted to the centralized system via Wi-Fi antennas 524 and 516.

In a different embodiment, it may be understood that instead of performing the analysis on-site using the sample storage and/or analysis unit 512 and the sample analysis unit 518, the samples may be collected and stored for analysis, so that collected and stored samples may be analyzed at a different site, such as a lab. Thus, for example, the robot 502 may collect and store the samples. The physical samples may be transported to the lab or may be picked up to be transported to the lab. And in further examples, the data associated with the samples may also be wirelessly transmitted to the centralized system transmitted via the Wi-Fi antennas for further analysis.

Regardless of the where and/or when the analysis is performed, it may be understood that the centralized system may receive, store, maintain, manage, and “know” at all times all data and information related to the analysis of all samples collected in the environment and performed monitoring assessments so as to at least send alerts to appropriate personnel in the event that contamination is detected or where corrective actions need to be initiated as part of overall improvement plans.

Moreover, it may be understood that some or all of the components of the robot 502 may be modular in that personnel can switch out, replace, add, subtract, etc. the components so as to customize the robot 502 suitable to the environment and the monitoring thereof. Additionally, the robot 502 may have various sensors that are configured to detect and/or monitor the components of the robot 502, for example, sensors that can detect whether samples are accidentally spilled while the robot 502 is moving or if air is leaking out of the air sampling unit 504.

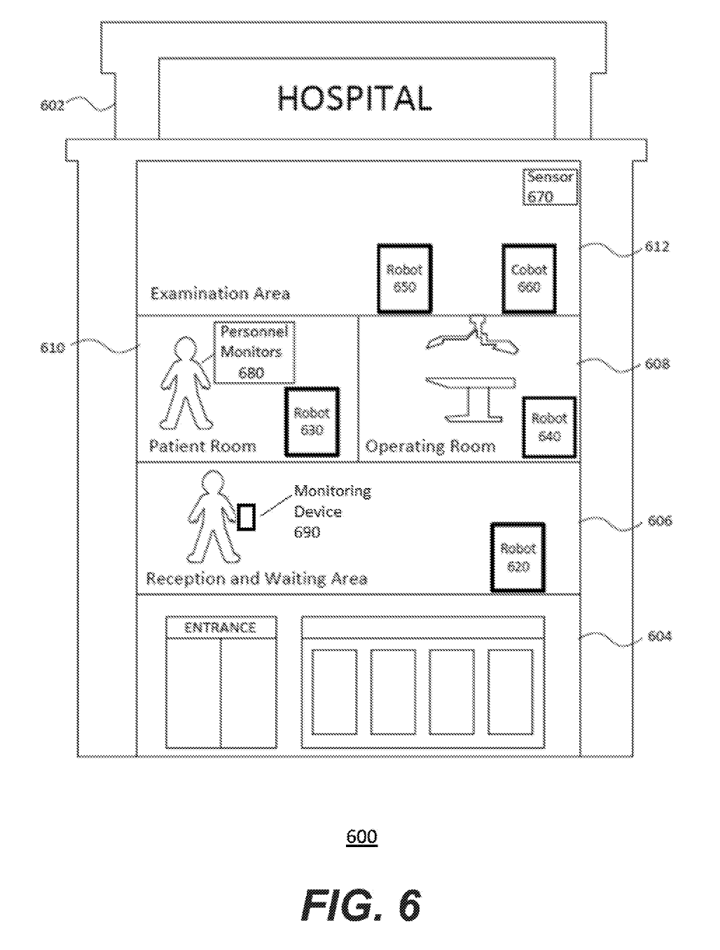

FIG. 6 illustrates an example of an environment 600 in accordance with one or more aspects of the present invention. As shown, the environment 600 is a hospital 602. Although only a single building is illustrated, the hospital can include a plurality of buildings in a plurality of locations, such as a single hospital with multiple buildings/departments/clinics located remotely from the main hospital building, or a chain of hospitals that fall under a single organization.

Hospital 602 may have multiple levels, for example, at least four levels as depicted in FIG. 6. A first level may include at least an entrance area 604. A second level may include at least a reception and waiting area 606. A third level may include at least an operating room 608 and a patient room 610. A fourth level may include at least an examination area 612.

Throughout the various locations of the hospital 602, there may be one or more robots and/or cobots and/or fixed sensors that collect (and optionally, analyze) numerous samples at those locations. There may also be handheld sampling devices 690 and/or monitors of personnel 680. By way of example, robot 620 is deployed in the reception and waiting area 606 and may collect samples from surfaces of one or more reception desks as well as samples of the air. By way of another example, robot 630 may be deployed in the patient room 630 and may swab or sample various surfaces in the room and perform ATP measurements. By way of a further example, robot 640 may be deployed in the operating room 608 prior to a surgery in order to detect any contamination on the operating surfaces, machinery, surgical instruments, tools, etc. By way of an additional example, robot 650 and cobot 660 may be deployed in the examination area 612 also monitoring that area for contamination. The cobot 660, for instance, may receive and store samples (e.g., swabs) from hospital personnel and transported to an analytics site, such a lab that may be several rooms down from the examination area 612. The robot 650 may be configured similarly to robots 620, 630, and 640 and automatically monitor the examination area 612. Any of these robots or cobots can also be used to check other robots or cobots for the presence of contaminants. Optionally, the robots or cobots could also have equipment to de-contaminate each other. In addition, sensors 670 can be mounted to test contaminants in water, the air, or the like and feed data directly into the robot 650 or the system.

The hospital 602 may implement a centralized system for managing the monitoring of the entire hospital environment. For example, the centralized system may be facilitated by one or more computing devices (e.g., server computers, virtual cloud computing devices, etc.) and one or more storage devices, all of which may be connected to a centralized hospital network. The robots 620, 630, 640, 650 and cobot 660, for instance, may receive from and transmit information to the network via various antennas (e.g., Wi-Fi antennas) that may be installed at various locations of the hospital 602, such as at least the entrance area 604, the reception and waiting area 606, the operating room 608, patient room 610, and the examination area 612.

The robots and cobots deployed in the hospital environment may directly receive instructions from the centralized system via the hospital network, such as routing information (e.g., moving from location to another location, how to move within a certain location), workflow and work related assignments (e.g., what samples to collect, where to collect, how many samples to collect, whether to perform the analysis on-board or to deliver the sample somewhere else for analysis, etc.), information that can be displayable by the robots or cobots to hospital personnel related to hospital monitoring (e.g., the robot or cobot can display “This area is clean and free of contamination,” “This area is quarantined,” or the like). Furthermore, the centralized system may automatically and dynamically generate additional routing algorithms, which may be based on changes in the environment or need, record the locations of the samplings, schedule the samplings (e.g., routine, scheduled, random), provide navigation information to robots or cobots, dynamically prioritize the samplings based things like real-time data, or past or forecasted contaminations, and may also code the samples in a manner suitable for processing.

Also, the system can be used to monitor environments after construction or renovation to ensure that all of the contaminants have been removed prior to opening or reopening those environments.

Additionally, the robots and cobots may transmit information to the centralized system, which may include results of the analysis of collected samples for report and alert generation, where the robots and cobots have already monitored (or where they have not monitored), how many samples they have already collected, any physical obstructions that may prevent the robots or cobots from moving within the environment according to respective routing instructions, any and all visual data captured by image or audio sensors (e.g., video, audio), information related to specific contaminations (e.g., whether they have been eradicated), the location of certain contaminated patients (e.g., if the patient has been moved from one patient room to a different room), etc.

In at least that regard, the centralized system of the hospital 602 may be able to dynamically maintain, update, perform further analysis on, generate reports, alerts, trends, forecasts, etc. on all information related to the monitoring of the hospital 602 in a streamlined, organized, singular, and efficient manner. The information may also include an inventory of eradication or testing consumable products, which allows hospital personnel to purchase more of those products. Based on this information, hospital personnel may be suggested or prompted to obtain certain eradication or testing consumable products. Additionally, the reports provided to the hospital enable more efficient completion of mandatory governmental reporting by the hospital.

The information on the centralized system may be accessed by authorized hospital personnel using mobile computing devices (e.g., smartphones, laptops, tablet computers) that are connected to the hospital network. As described above, FIG. 1 may represent the components and functionalities of the centralized system implemented in the hospital 602. Moreover, it may be understood that the hospital network, which may be a local network, may also be connected to one or more external networks, such as the Internet, cloud-computing networks, etc. It may be further understood that existing and new equipment may be added to or removed from the hospital network.

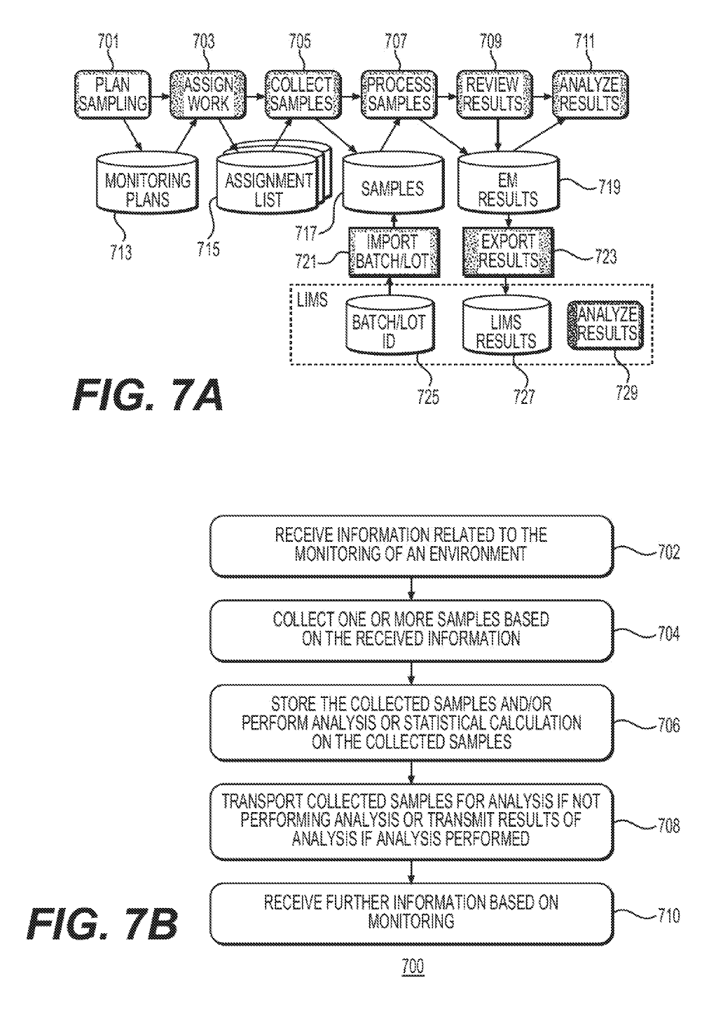

FIG. 7A illustrates a flow of how a system may monitor a particular environment in accordance with one or more aspects of the present invention.

For example, sampling plans may be generated in step 701, which in conjunction with monitoring plans generated in step 713, can be used to create work assignments in step 703 for one or more robots and/or cobots and/or personnel in the environment. The work assignments may be used to create an assignment list in step 715 that can be used to generate work assignments that are sent to the respective robots and/or cobots and/or personnel.

An environment monitoring assessment may be conducted and samples may be collected in step 705 and stored in step 717 within the environment and analysis on the collected samples may be performed in step 707. The monitoring may include a continuous and/or on-demand data feed of environmental parameters, such as temperature, humidity, air flow, etc. These parameters are important for optimizing patient care, particularly in high risk patient rooms and operating theaters. Refrigerators and freezers can be fitted with sensors that continuously monitor the temperature, such that this data could be collected in the system and alerts sent if they go outside of agreed ranges. Temperature monitoring of equipment such as refrigerators and freezers is necessary where medicines like antibiotics and vaccines need to be stored in controlled temperature conditions. The results of the analysis may be stored in one or more databases (e.g., “EM Results”) in step 719, where the results can be reviewed in step 709 and analyzed in step 711 by users. For example, compliance statistics can be calculated for the collected data. The results of the analysis may be output in step 723 in any desirable form (e.g., dashboard, alerts, reports, trends, forecasts, etc.) to personnel and/or EHR and/or EMR systems in a timely manner. In step 727, a laboratory information management system (LIMS) receives the outputted results and analyzes the results in step 729. Further, the LIMS can store batch/lot ID information in step 725, which is output in step 721 to facilitate processing of sample in step 707.

FIG. 7B illustrates a flow chart 700 from the perspective of a robot or cobot and/or personnel monitoring an environment in accordance with one or more aspects of the present invention.

In step 702, the robot or cobot may receive information related to the monitoring of the environment. The information may include routing information related to how to navigate within and among the various locations within the environment and/or work assignments.

Based on the received information, the robot or cobot and/or personnel may collect one or more samples and/or monitor environmental parameters such as temperature, humidity, air flow, etc. at step 704. The samples may include, for example, samples related to food hygiene, water testing, instrument sterilization/monitoring, hand hygiene, compounding pharmacies, clean room air quality, and the like. With respect to food hygiene, the cleanliness and microbial contamination of the environment/equipment plus fridge/freezer temperatures within food preparation areas (e.g., canteen, kitchen) of hospitals and other locations can be monitored. As described above, the water testing may include testing of facility potable water for temperature, Legionella, Pseudomonas aeruginosa, as well as the various use patterns that are tested from an Infection Prevention & Control perspective such as endoscope rinse water, renal dialysis water, hydrotherapy pools, etc. Instrument sterilization/disinfection may include, for example, collecting all data relating to the cleaning, disinfection and sterilization of instruments and mobile hospital equipment. Further, the system is able to scan a bar code and/or radio-frequency identification (RFID) tag associated with each instrument or piece of equipment (e.g., bed, wheelchair, monitoring equipment) and link the sampling/monitoring time with the data, including all parameters connected to the reprocessing as well as any cleaning verification/monitoring data, such as when the object was cleaned/disinfected/sterilized along with a personnel number/equipment/chemicals used plus any verification/monitoring checks and any associated data from the EM conducted in a Central Services Sterilization Department (CSSD) processing room. The collected data can be output to other IT systems such as EHR/EMR systems. For compounding pharmacies the system can satisfy the legal requirements to monitor all aspects of the environment, equipment and workers in cleanrooms and pharmacy facilities within a hospital that manufacture personalised medicines and cell therapies. The samples and environmental data may be stored at step 706. If the robot or cobot is configured with analytics or testing equipment, it may also take the collected samples and/or environmental data and perform analysis or statistical calculation at step 706. Additionally, step 706 may include the robot or cobot cleaning or communicating the need for cleaning of the sampled site to site systems or personnel.

In step 708, the robot or cobot or personnel may either transport the collected and stored samples to a different site or equipment for further processing (if the robot or cobot is not equipped with onboard analytics equipment) or the robot or cobot or personnel may transmit (e.g., wirelessly) the results of the analysis to a centralized system (e.g., to one or more computing devices of the system) (if the robot or cobot performs the analysis itself).

In step 710, the robot or cobot or personnel may receive further information based on the results of the analysis. For example, if there is a contaminant detected, the robot or cobot or personnel may be further instructed to eradicate the contamination (if equipped with cleaning agents or cleaning equipment), or the robot or cobot may be further instructed to find the closest environment personnel and warn the personnel of the contamination, or it may be further instructed to display the warning or alert on a display interface (if the robot or cobot has a display interface or on another device), etc.

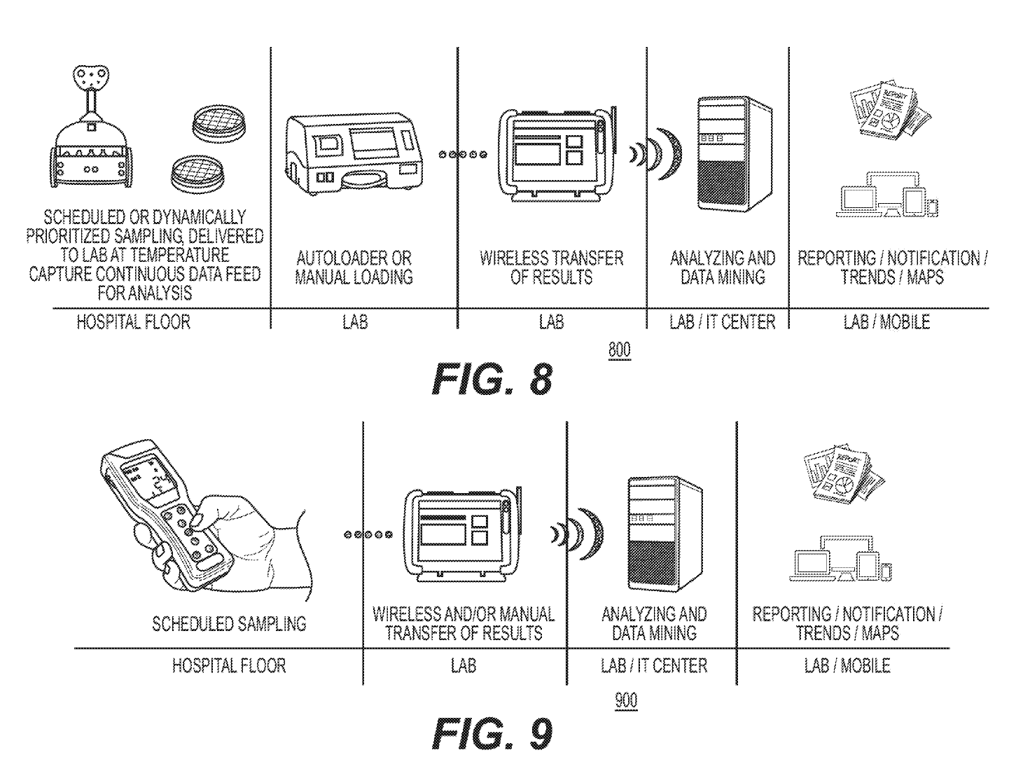

FIG. 8 illustrates a contact plate method 800 for monitoring an environment in accordance with one or more aspects of the present invention.

By way of example, a robot may receive instructions to monitor the environment using contact plate analysis. For instance, contact plates may be used for cultivation of microorganisms from environmental surfaces. Each contact plate may have a specified grid molded into the plate and the plate may further have a friction lid that ensures proper sealing. The robot may take a plate, using robotic arms and respective end effectors, and press the plate down firmly on the test surface, such as a surface on a hospital floor. Rolling motions may be used for curved surfaces then perform or schedule cleaning thereafter.

After sample collection, the robot may seal the plate with friction lid and store the plate in an incubator for a prescribed period of time. As described above, the incubator may be configured as a component of the robot.

In the example shown in FIG. 8, the robot may transport the plate to a lab, where the plate can be inserted or placed into an analytics equipment or testing equipment, such as an autoloader or manual loading device, as illustrated, for analysis of the collected surface sample. It may be understood, that the analytics equipment may also be a component of the robot, in which case the robot would not be required to transport the sample to a lab.

The results of the analysis of the collected sample may then be wirelessly transferred or transmitted to a centralized system (e.g., rack mounted computing devices in an IT center or lab), where the results can be further analyzed. And based on those results (and based on the further analysis if performed), various reports, notifications, alerts, trends, forecasts, maps, etc. may be generated by the system.

FIG. 9 illustrates an ATP measurement method 900 for monitoring an environment in accordance with one or more aspects of the present invention.

By way of example, a robot may receive instructions to monitor the environment using ATP measurement analysis. For instance, an ATP measurement device may be used, which is designed to rapidly measure the amount of contamination on any surface. While a hand is shown in FIG. 9 holding the ATP measurement device, it should be understood that the robot may be holding such device to perform monitoring or a similar device may be incorporated or configured in the robot as a component or transported by a cobot for use by a human.

A swab may be used by the robot to collect samples from an area of a surface. The robot may insert the swab into a swab tube, where a liquid reagent can be released into the tube. The swab tube may then be inserted into the ATP measurement device, where it will measure the amount of adenosine triphosphate (ATP) from the swab.

The ATP reading from the measurement device may be directly and wirelessly transmitted to a centralized system (e.g., rack mounted computing devices in an IT center or lab) or manually entered into the system. The results, similar to FIG. 8, may be used to generate reports, notifications, alerts, trends, forecasts, maps, etc. It may be understood that the ATP measurement analysis can be performed by the robot itself or the robot may transport the swab to a lab where the ATP measurement can be obtained.

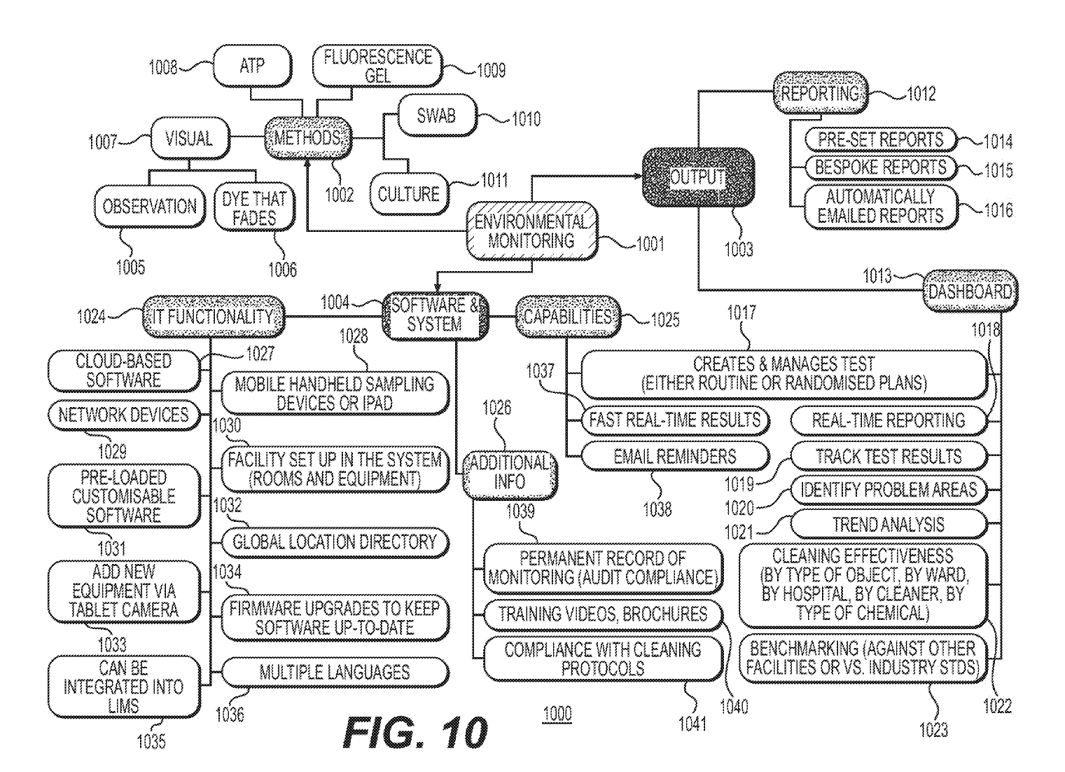

FIG. 10 illustrates an environmental monitoring diagram in accordance with one or more aspects of the present disclosure.

As shown, various functions of environmental monitoring may include a centralized system and associated software with various IT functionalities (e.g., cloud based software, network devices, pre-loaded customizable software, new equipment additions, integration, mobile handheld sampling devices or tablet computers, facility set up, global location directory, firmware upgrades, multiple language features), multiple capabilities (e.g., create and manage tests, fast real-time results, e-mail reminders), and additional features (e.g., permanent record of monitoring for things like audit compliance, training videos, brochures, compliance with cleaning protocols), and the monitoring may further include various outputs, such as reporting (e.g., pre-set reports, bespoke reports, automatically e-mailed reports) and may be output via different interfaces like a dashboard (e.g., real-time reporting, track test results, identify problem areas in the environment, trend analysis, cleaning effectiveness by type of object, by ward, by hospital, by cleaner, by type of chemical used, etc., benchmarking against other facilities or industry standards). It can also detect antimicrobial resistance based on the proportion of various contaminants in each area and the change in that proportion over time.

As illustrated in FIG. 10, the environmental monitoring diagram 1000 for environmental monitoring 1001 includes a plurality of different methods 1002 and outputs 1003 and may be implemented in a variety of software and systems 1004. The methods of environmental monitoring include, for example, observation 1005, dye that fades 1006, visual 1007, ATP 1008, fluorescence gel 1009, swab 1010, and culture 1011. The outputs 1003 of the system include, for example, reporting 1012 and dashboard 1013. The reporting 1012 may include, for example, pre-set reports 1014, bespoke reports 1015, and automatically emailed reports 1016. The dashboard 1013 may include, for example, creating and managing tests 1017, real time reporting 1018, track test results 1019, identify problem areas 1020, trend analysis 1021, cleaning effectiveness 1022 (e.g., by type of object, ward, hospital, cleaner, chemical), and benchmarking 1023 (e.g., against other facilities or industry standards).

The software and system 1004 illustrated in FIG. 10 may include, for example, IT functionality 1024, capabilities 1025, and additional information 1026. The IT functionality 1024 may include, for example, cloud-based software 1027, mobile handheld sampling devices 1028, network devices 1029, facility set up in the system 1030, pre-loaded customizable software 1031, global location directory 1032, new equipment 1033, firmware upgrades 1034, LIMS integration 1035, and multiple languages 1036. The capabilities 1025 of the software and system 1004 may include, for example, creating and managing tests 1017, fast real-time results 1037, and email reminders 1038. The additional information 1026 of the software and system 1004 may include, for example, a permanent record of monitoring 1039 for audit compliance, training videos and brochures 1040, and compliance with cleaning protocols 1041.

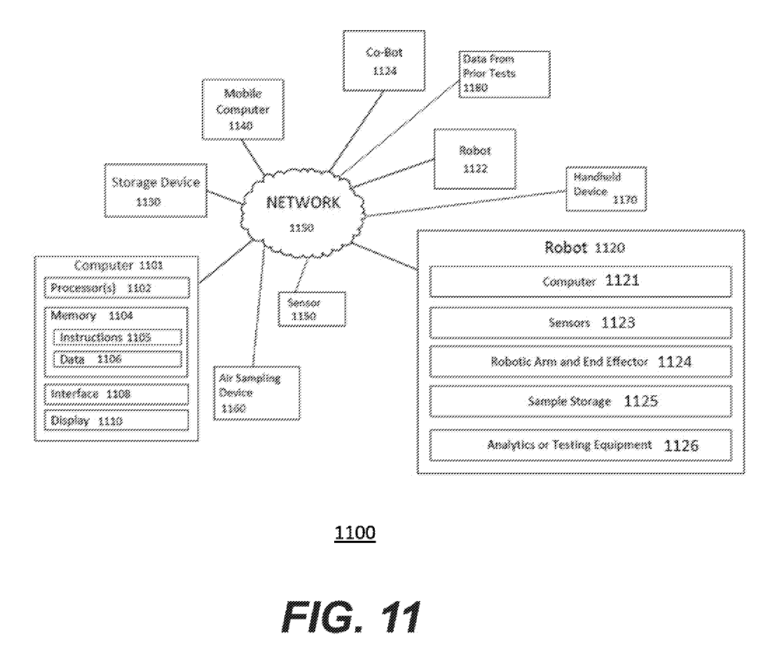

FIG. 11 illustrates an example system 1100, which may be implemented within an environment, such as a hospital, in accordance with one or more aspects of the invention. As shown, the system 1100 includes at least a computer 1101, robots 1120 and 1122, cobot 1124, a storage device 1130, a mobile computer 1140, a sensor 1150, an air sampling device 1160, which may all connect to a network 1150 (as depicted by the dashed lines) and communicate with each other or other devices either on the same network or other networks.

For example, computer 1101 includes one or more processors 1102, memory 1104, e.g., permanent or flash memory (which includes instructions 1105 and data 1106), an interface 1108, and a display 1110.

Processor 1102 may instruct the various components of the computer 1101 to perform tasks based on the processing of certain information, such as instructions 1105 and/or data 1106 stored in the memory 1104. The processor 1102 may be any standard processor, such as a central processing unit (CPU), or may be a dedicated processor, such as an application-specific integrated circuit (ASIC) or a field programmable gate array (FPGA) or an industrial process controller or the like.

Memory 1104, whether permanent or flash, may be any type of hardware (e.g., ROM, RAM, CD-ROM, hard drive, write-capable, read-only, etc.) configured to store information accessible by the processor 1102, such as instructions 1105 and data 1106, which can be executed, retrieved, manipulated, and/or stored by the processor 1102. The instructions 1105 stored in memory 1104 may include any set of instructions (e.g., “steps” or “algorithm” associated with software) that can be executed directly or indirectly by the processor 1102. The data 1106 stored in memory 1104 may be retrieved, stored or modified by the processor 1102, for example, in accordance with the instructions 1105.

Interface 1108 may be a particular device for interfacing with the computer 1101 (e.g., a field-mounted instrument, processor-to-processor communication, keyboard, mouse, touch sensitive screen, camera, microphone, etc.), a connection or port (e.g., data port, USB, zip drive, card reader, CD driver, DVD drive, etc.), and/or software (e.g., graphical user interface) that allows the reception of information and data.

Display 1110 may be any suitable type of device capable of communicating data to a user, such as liquid-crystal display (LCD), light emitting diode (LED), and plasma screens.

Robot 1120 may be an autonomous, self-navigating robot configured to move within an environment to collect, process, and/or perform analysis on the different samples and may be wirelessly connected to the network 1150 so as to transmit/receive various information and data over the network. Robot 1120 may include a computer 1122, sensors 1124, one or more robotic arms and respective end effectors 1126, and sample analytics and/or testing equipment 1128.

Computer 1122 of robot 1120 may be configured similarly or identically to the above-described computer 1101 and include one or more processors, memory, an interface, and display. The computer 1122 may receive routing or navigation instructions over the network 1150 and move the robot 1120 according to those instructions.

Sensors 1124 may include image sensors, laser sensors, infrared sensors, touch-sensitive sensors, acoustic sensors, or any other type of sensor configured to guide the robot 1120 or assist in various robotic functionalities. For example, one or more of the sensors 1124 may be configured to assist the robot 1120 detect various obstacles in the environment and react accordingly, such as dodging or weaving around the obstacles. In some embodiments, the robot 1120 may have a badge scanner in order to scan personnel badges in the environment. In at least that regard, the robot 1120 may be able to determine at least two things: whether the personnel is or is not supposed to be at that particular location of the environment and also whether the personnel is contaminating the environment, and if so, alerting the personnel of such contamination (e.g., telling the personnel to wash his or her hands, telling the personnel that he or she needs a refresher on handwashing practices).

Robot 1120 may further include at least one robotic arm with a respective end effector, such as a robotic hand, gripper, tool, sensor, etc., for use in collecting the sample(s), which may be stored in the sample storage 1125 until the robot 1120 can deliver the sample(s) to testing equipment or a lab for analysis. Additionally and/or alternatively, the robot 1120 may process and perform analysis on the sample(s) itself with onboard analytics or testing equipment 1126, which will be further described below. Additionally, robot 1120 can collect data from other robots. Robot 1120 can also test the quality of the cleaning performed by personnel or other robots, such as ultra-violet emitting (UV) robots or Vaporous Hydrogen Peroxide (VHP) robots.

The system 1100 may include other robots, such as robot 1122. Robot 1122 may be configured similarly or identically to robot 1120 and may collect samples different than the samples collected by robot 1120. Moreover, robot 1122 may include testing equipment different than the analytics or testing equipment 1126 of robot 1120. To at least that end, robot 1122 may be deployed in a different part of the environment than robot 1120. And similar to robot 1120, robot 1122 may also receive routing or navigation instructions over a network 1150—but not necessarily the same network—and also transmit/receive various information and data over the network.

The system 1100 may also include one or more cobots, such as cobot 1124, which may be different from a robot in that it may be semi-automated and not fully automated like a robot. For example, cobot 1124 may assist personnel, such as a hospital lab technician or employee, receive and store samples that were manually collected by the personnel. Thus, the collection of the samples may not be automatically performed by the cobot 1124. In that way, the cobot 1124 may only assist the user to perform a particular task. Moreover, cobot 1124 may include the same types of components (e.g., computer, sensors, arm and end effector, storage, etc.) that are also included in the robots 1120 and 1122. Moreover, the cobot 1124 may be moveable on its own or may be physically moved (e.g., pushed, pulled) by the user.

The storage device 1130 may be configured to store a large quantity of data and may also be configured to transfer such data when requested or accessed by other components of the system 1100, either through the network 1150 or otherwise. For example, the storage device 1130 may be a collection of storage components, such as ROM, RAM, hard-drives, solid-state drives, removable drives, network storage, virtual memory, multi-leveled cache, registers, CD, DVD, etc. In addition, the storage device 1130 may be configured so other components of system 1100, such as the computer 1101, robots 1120 and 1122, cobot 1124, and/or mobile computer 1140 can have access and provide data to it.

In embodiments, the system 1100 may be implemented as a single, centralized system in the environment to facilitate the monitoring of the environment. The “brains” of the system may be carried out by one or more computers, such as computer 1101, which may be server computers that reside physically at the environment, or elsewhere. The computer 1101 may provide instructions to robots 1120 and 1122 and cobot 1124 via antennas installed throughout the environment. In some embodiments, the robots can communicate directly with each other using electronic, laser, ultrasonic or other means.

Additionally, information and data may be received by the computer 1101 from the robots and cobots and sensors and personnel operated devices in the system, which may include data related to the various samples that have been collected and analyzed from the environment. In examples, the system may send alerts to various computing devices, such as the mobile computer 1140 (which may belong to hospital personnel). Moreover, the system may account for Wi-Fi gaps or equipment interference with Wi-Fi communication, and the system may also be configured to operate offline. In at least that manner, the system is robust. As will be further described below, the “brains” of the system may regulate various tasks for the robots and cobots, such as generating, maintaining, and updating routing and navigation instructions, dynamically setting up schedules for various types of testing, keeping track of infected or contaminated locations and/or people in the environment, and dynamically updating its respective database and any and all information related to the monitoring of the environment.

It may be understood that the aforementioned features may also be applied in any one of the examples and embodiments described above.

As discussed above, the analytics used for monitoring the environment includes ATP, gel-based methods, swabbing techniques, culture based methods, visual methods, such as observation or using fading dyes.

The present invention is advantageous in various ways. For example, the present invention allows the monitoring of an environment to be integrated into a single, centralized system, a system which may employ one or more robots and/or one or more cobots to collect various samples within the environment and perform analysis on the samples so that a contamination can be detected quickly and in real-time. Detection of a contamination triggers an automatic response by the system so that appropriate environment personnel may eradicate the contamination in a timely fashion or generate data that can support long term improvement plans. Moreover, the robots or cobots themselves may be equipped with cleaning agents or cleaning devices (such as UV light) to eradicate the contamination or perform post-human-cleaning efficacy sampling.

The present invention is also advantageous because the centralized system may dynamically maintain, organize, and update all information related to all monitoring of the environment in one place. Thus, the end result is a more streamlined way of accessing the data, for example, a dashboard interface may output a report, analysis, trends, forecasts, maps, notifications, alerts, updates, etc. to a user, such as the environment personnel, in order to ensure the environment is being properly monitored. The system may perform dynamic and responsive scheduling based on need and the changing environment (e.g., schedule additional sampling via robot or personnel, send more robots to a certain area if contamination is detected, focus on the monitoring of specific areas of the environment if a contaminated object or person moves from one location to another). Moreover, the present invention is advantageous because sampling results are obtained quickly. The quicker the results are obtained, the faster the system can respond to possible contaminations, thereby saving time and make more efficient the monitoring process compared to the prior art. Given the logarithmic growth or spread of contaminants, a significant time reduction is a significant improvement over the prior art.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof. Although the disclosure uses terminology and acronyms that may not be familiar to the layperson, those skilled in the art will be familiar with the terminology and acronyms used herein.

What is claimed is:

1. A system for performing analysis comprising:
at least one processor for executing stored program instructions to:
dynamically schedule collection of samples within a healthcare environment, monitoring assessments within the healthcare environment, or other data relating to the healthcare environment;
collect or coordinate collection of the samples, the monitoring assessments, or the other data relating to the healthcare environment;
process or coordinate processing of at least one of collected samples and input of monitoring results;
perform analysis on or coordinate performance of analysis on the collected samples;
determine or detect whether any contaminants are present in the healthcare environment based on at least one of the analysis of the collected samples and the monitoring results; and
based on a determination or a detection of the contaminants, automatically output information.

2. The system of claim 1, wherein the samples are collected by a robot.

3. The system of claim 1, wherein the samples are collected or the monitoring is conducted by one or more of the following: a cobot, a robot, and personnel.

4. The system of claim 1, wherein the analysis is performed by one or more of the following: a cobot, a robot, and personnel.

5. A system for monitoring a healthcare environment, the system comprising:
at least one computing device for executing stored instructions to:
send at least one of routing instructions and work instructions;
receive results of an analysis on a sample or collection of monitoring assessment data of the sample;
determine whether a location of the healthcare environment contains a contamination; and
generate an output based on a determination of the contamination;
wherein the output includes instructions to a user to remediate the contamination.

6. A system for monitoring a healthcare environment, the system comprising:
one or more robots configured to:
move automatically in the healthcare environment,
collect a sample from a location of the healthcare environment,
at least one of store and perform an analysis on a collected sample, and
clean or output a notification of a need for cleaning based on the analysis or results from sampling; and
a computing device for executing stored instructions to:
send at least one of routing instructions and work instructions to the one or more robots;
receive results of the analysis on the collected sample;
determine whether the location of the healthcare environment contains a contamination; and
generate an output based on a determination of the contamination.

7. The system of claim 6, wherein the healthcare environment is a hospital.

8. The system of claim 6, wherein the one or more robots includes one or more of the following: (i) a computer, (ii) a sensor, (iii) a robotic arm, (iv) an end effector, (v) an incubator, (vi) analytics or testing equipment, and (vii) a self-navigating vehicle, base, or platform.

9. The system of claim 6, wherein the at least one sample includes one or more of the following: (i) a sample of a surface in the location of the healthcare environment, (ii) a sample of air in the location of the healthcare environment, (iii) a sample of water in the location of the healthcare environment, (iv) a sample taken from a human, person, or personnel in the location of the healthcare environment, (v) physical measurement of the healthcare environment, and (vi) observation of the healthcare environment.

10. The system of claim 6, wherein the analysis performed on the collected sample is based on one or more of the following: (i) a visual system, (ii) ATP measurement, (iii) a marking agent, (iv) contact plate, (v) rapid detection method, (vi) next generation sequencing (NGS), (vii) whole genome sequencing (WGS), (viii) organism identification, (ix) viable count, (x) quick Polymerase Chain Reaction (qPCR), (xi) total cell count, (xii) batch scanners, and (xiii) comparison of at least one of collected temperature, humidity and air flow monitoring data against predetermined limits.

11. The system of claim 10, wherein the visual system includes use of at least one of visual checklists, fading dyes, fluorescent gels with UV light detector, and an image sensor.

12. The system of claim 6, wherein the routing instructions include instructions for the one or more robots to move from a first location to a second location in the healthcare environment.

13. The system of claim 1, wherein the information is an alert when the contamination is detected, the alert being an e-mail or text alert sent to one or more mobile computing devices connected to a network of the system.

14. The system of claim 1, further comprising one or more cobots.

15. The system of claim 1, wherein the computing device is configured to provide in real-time a dashboard, a report, an alert, trend analysis, and a forecast, and allow for data mining based on all data associated with the monitoring of the healthcare environment.

16. A device for monitoring a healthcare environment, the device comprising:
one or more sensors;
a self-navigating vehicle, base, or platform;
at least one tool;
sample storage; and
at least one computing device for executing stored instructions to:
collect at least one sample from the healthcare environment; and
store a collected sample in the sample storage.

17. The device of claim 16, wherein the device is a robot or a cobot.

18. The device of claim 16, wherein the at least one tool includes a robotic arm and an end effector.

19. The device of claim 16, wherein the sample storage is an incubator.

20. The device of claim 16, further comprising analytics equipment.

21. The device of claim 20, wherein the at least one computing device is configured to perform analysis on the collected sample using the analytics equipment.

22. The device of claim 21, wherein the analysis performed on the collected sample is based on one or more of the following: (i) a visual system, (ii) ATP measurement, (iii) a marking agent, (iv) contact plate, (v) rapid detection method, (vi) next generation sequencing (NGS), (vii) whole genome sequencing (WGS), (viii) organism identification, (ix) viable count, (x) quick Polymerase Chain Reaction (qPCR), (xi) total cell count, and (xii) comparison of at least one of collected temperature, humidity and air flow monitoring data against predetermined limits.

23. The device of claim 22, wherein results of the analysis on the collected sample is sent to a system via at least one antenna connected to a network.

24. The device of claim 16, wherein the device is configured to transport the collected sample to testing equipment or to a location where an analysis on the collected sample is performed.

25. A method for monitoring an environment, the method comprising:
sending, by at least one computing device, at least one of routing instructions and work instructions to one or more robots, wherein one or more robots are configured to move automatically within the environment, collect at least one sample from a location of the environment, and at least one of store and perform an analysis on a collected sample;
receiving, by the at least one computing device, results of the analysis on the collected sample or input of an observational monitoring assessment; and
determining, by the at least one computing device, whether the location of the environment contains a contamination; and
generating, by the at least one computing device, an output based on a determination of the contamination.

26. A method for monitoring an environment, the method comprising:
collecting, using at least one tool, a sample from the environment;
storing a collected sample in a sample storage; and at least one of:
(i) transporting, using a self-navigating vehicle, base, or platform, the collected sample to testing equipment or to a location where an analysis on the collected sample is performed; and
(ii) performing the analysis, using analytics equipment, on the collected sample.

27. The system of claim 1, further comprising one or more sensors for monitoring hand hygiene, wherein the at least one processor is configured to: receive data via the one or more sensors, determine whether a contaminant is present on a hand of a user, and automatically generate an alert to the user based on the determination.

28. The system of claim 1, wherein the at least one sample includes one or more of the following: (i) a sample of a surface in the location of the healthcare environment, (ii) a sample of air in the location of the healthcare environment, (iii) a sample of water in the location of the healthcare environment, (iv) a sample taken from a human, person, or personnel in the location of the healthcare environment, (v) physical measurement of the healthcare environment, and (vi) observation of the healthcare environment.

29. The system of claim 1, wherein the analysis performed on the collected sample is based on one or more of the following: (i) a visual system, (ii) ATP measurement, (iii) a marking agent, (iv) contact plate, (v) rapid detection method, (vi) next generation sequencing (NGS), (vii) whole genome sequencing (WGS), (viii) organism identification, (ix) viable count, (x) quick Polymerase Chain Reaction (qPCR), (xi) total cell count, (xii) batch scanners, and (xiii) comparison of at least one of collected temperature, humidity and air flow monitoring data against predetermined limits.

30. The system of claim 29, wherein the visual system includes use of at least one of visual checklists, fading dyes, fluorescent gels with UV light detector, and an image sensor.

31. The system of claim 1, wherein the healthcare environment is a hospital.

32. The system of claim 1, wherein the samples are collected from at least one of surfaces, air, water, and personnel.

33. The system of claim 1, wherein the information includes at least one of an alert, a report, a trend, and a forecast.

34. The system of claim 1, wherein the information is outputted to at least one of an environmental service, a facilities management service, a cleaning company, infection prevention personnel, an electronic health record system, an electronic medical record system and a governmental agency.

35. The system of claim 1, wherein the at least one processor dynamically schedules the collection of samples based on a determination of needs and changes in the healthcare environment.

36. The system of claim 1, wherein the healthcare environment includes a plurality of healthcare environments.

37. The system of claim 1, wherein the at least one processor executes stored program instructions to compare the determination or the detection of the contaminants with other system data to verify that a contamination exists prior to outputting the information.
